(19) United States
(12) Patent Application Publication
(10) Pub. No.: US 2026/0152819 A1
(43) Pub. Date: Jun. 4, 2026

(54) PROCESS

(71) Applicant: Cemvita Factory, Inc., Houston, TX (US)

(72) Inventors: Stephan CHRISTEL, Westminster, CO (US); Marny REAKES, Westminster, CO (US); Tom CLARK, HOUSTON, TX (US); Jayme FEYHL-BUSKA, Westminster, CO (US); Jillian AYERS, Westminster, CO (US); Colbey DERWIN, Westminster, CO (US)

(21) Appl. No.: 18/672,476

(22) PCT Filed: Feb. 23, 2024
(86) PCT No.: PCT/US2024/017036

§ 371 Date: May 23, 2024

Related U.S. Application Data
(63) Continuation-in-part of application No. 18/174,170, filed on Feb. 24, 2023.

(30) Foreign Application Priority Data
May 10, 2023 (GB) 2306877.8
Nov. 13, 2023 (GB) 2317389.1

(51) Int. Cl. C22B 3/18; C12M 1/34; C12N 1/20; C12R 1/01

(52) U.S. Cl. CPC C22B 3/18; C12M 41/12; C12M 41/46; C12N 1/20; C12R 2001/01

(57) ABSTRACT

A process comprising: i) providing a body of metal ore, ii) contacting the body of metal ore with a microbial lixiviant comprising one or more populations of microorganisms, iii) measuring at least one parameter indicative of function of the one or more populations of microorganisms, and iv) adjusting one or more process variables in response to the measured parameter.

This application is a continuation-in-part application of U.S. patent application Ser. No. 18/174,170, filed Feb. 24, 2023. This application also claims priority of International Application No. PCT/US24/17036, filed Feb. 23, 2024, which claims priority from U.S. Non-provisional application Ser. No. 18/174,170, filed Feb. 24, 2023, GB Application No. 2306877.8, filed May 10, 2023, and GB Application No. 2317389.1, filed Nov. 13, 2023. The entirety of all of the aforementioned applications is incorporated herein by reference.

### FIELD

The present invention provides metal leaching processes as well as compositions useful in such processes.

### BACKGROUND

The extraction of metals and minerals from the earth has been of importance to humanity for millennia. Techniques for doing so have evolved over time. In recent years, there have been growing concerns regarding the environmental impact of mining metal ores and extracting the metal of interest therefrom. Mining, tunnelling, and blasting have profound effects on the physical environment. Transport and extraction of metals from ore introduce toxic chemicals into the environment and utilize significant amounts of energy.

It is therefore not surprising that steps have been taken to minimize the environmental impact of extracting metal from ore. One such approach is leaching followed by solvent extraction/electrowinning (known as SX/EW), which has been primarily used to obtain copper and gold, as well as other metals such as uranium, zinc, and nickel.

With such leaching approaches, ore containing the mineral of interest is exposed to a lixiviant (e.g., an acid such as sulfuric acid) to leach out the metal of interest. The metal from the metal-rich leachate (or pregnant liquor solution) is then extracted from the leachate using an organic solvent (solvent extraction) and then transferred to an electrolyte. The electrolyte is then electrolyzed, forming the metal of interest in a pure form (i.e., electrowinning). Examples of leaching processes are disclosed in Australian Patent Application No. 2004240193, European Patent Publication No. 2024523 and International Patent Publication No. WO02/081761.

There are multiple ways in which such leaching approaches have been deployed in practice.

One such method of deploying the leaching approach is via dump or heap leaching processes. In these methods, ore is mined, crushed into smaller particulate form, and then amassed into a dump or heap. The dump or heap of particulate ore then has leaching lixiviant applied to it (e.g., by pouring or spraying). The lixiviant then flows through the dump or heap, leaching out the metal of interest, and the metal-rich leachate is then extracted from the base of the heap or dump.

A second example of how the leaching approach can be deployed is via tank-leaching—which is similar in approach to dump or heap leaching in that ore is firstly mined and crushed into particulate form. However, rather than a dump or heap of the particulate ore then being formed, the particulate ore is instead transferred to a tank or container containing the lixiviant. Such an approach may be preferable to dump or heap leaching where the metal of interest is particularly refractory to leaching. Additionally, the lixiviant is contained in a controlled environment meaning that the conditions in which the leaching step is performed can be optimized, and additionally the risk of inadvertent loss of solution to the environment is reduced.

One further example is in situ leaching. As the name suggests, the leaching step is conducted in situ, i.e., without having to firstly mine, tunnel, or blast out ore and then transport it for processing.

While leaching techniques have been successfully commercially deployed, attempts to improve their efficiency have been made. One such approach has been to employ lixiviants comprising bacteria, for example bacteria which are capable of metabolizing sulfur and/or iron. As those skilled in the art will be aware, minerals containing sulfur and iron are significant components of metal-containing ores. By exposing such ores to bacteria which are capable of metabolizing sulfur and/or iron, these minerals can be broken up and released into solution, thus freeing up the entrapped metal for leaching by the lixiviant.

Such ‘bioleaching’ processes have been commercially employed to extract copper and gold, and to a lesser extent uranium, zinc, and nickel. Research is also currently underway to explore the applicability of bioleaching to the extraction of rare earth elements. Thus, bioleaching has wide theoretical applicability.

Examples of bioleaching processes are known. For example, US 2008/0127779 discloses a process to increase the bioleaching speed of ores or concentrates of sulfide metal species by means of continuous inoculation with leaching solution that contains isolated microorganisms, with or without the presence of native microorganisms. US 2011/0045581 discloses a bioreactor for continuous production of bioleaching solutions for inoculation and irrigation of sulfide-ore bioleaching heaps and dumps.

However, one issue with conventional bioleaching applications is that following a period in which the microbial composition functions effectively to leach metal from the ore, a marked reduction in activity occurs. As and when a reduction in leaching efficiency is noted, fresh bacterial lixiviant is applied in an attempt to maintain or revive leaching activity. While this approach can be effective, the successful revival in leaching activity is at best temporary, necessitating repeated applications of bacterial lixiviant which adds to cost and complexity. Frequently, a completed revival in leaching efficiency with only a partial restoration being observed, if at all. In order to exploit the full potential of bioleaching, there is a need for more efficient processes and/or improved lixiviant compositions.

### SUMMARY

Thus, according to a first aspect of the invention, there is provided a process comprising: i) providing a body of metal ore, ii) contacting the body of metal ore with a microbial lixiviant comprising one or more populations of microorganisms, iii) measuring at least one parameter indicative of function of the one or more populations of microorganisms, and iv) adjusting one or more process variables in response to the measured parameter.

The inventors have identified that a shortcoming of conventional bioleaching methods is that the approach of periodically replenishing bacterial lixiviant results in inefficient bioleaching performance. By monitoring the function of the microbial lixiviant through the measurement of one or more parameters, this advantageously permits optimized bioleaching performance in terms of the functional lifespan of the lixiviant and/or the amounts of metal that can be recovered.

One benefit of the process of the present invention is its versatility. The bioleaching method can be applied to different leaching scenarios. Thus, in one embodiment, the body of metal ore is a heap optionally in proximity to the site from which the ore was mined. In such embodiments, the step of contacting the ore with the microbial lixiviant may be conducted by supplying the microbial lixiviant to the heap. In embodiments, the microbial lixiviant may be supplied to the heap at one or more locations on or in the heap. Additionally or alternatively, microbial lixiviant may be supplied to the heap on its surface or within the body of the heap. Any apparatus used to supply lixiviant to heaps may be utilized.

In certain embodiments, microbial lixiviant may be supplied to the heap during its assembly, i.e. in the heap stacking phase.

As used herein, the term “heap” is to be interpreted broadly to encompass conventional leaching heaps and dumps as well as other structures of metal ore which are capable of being leached.

Additionally or alternatively, the body of metal ore may be provided in a tank. In such embodiments, the step of contacting the metal ore with the microbial lixiviant may be conducted by supplying the microbial lixiviant into the tank.

In addition to versatility regarding the scenarios in which the process of the invention may be utilized, it may also be employed to extract a wide range of metals from ores. In embodiments of the invention, the metal ore may comprise copper. Examples of copper-containing ores which may be treated using the process of the present invention include chalcopyrite, cuprite, covellite, chalcocite, bornite, malachite, azurite, antlerite, tetrahedrite, chrysocolla, tennantite, enargite atacamite, brochantite, dioptase, rosasite, digenite, dioptase, tenorite, pseudomalachite, heterogenite, kolwezite and/or turquoise.

Additionally or alternatively, the metal ore may comprise of minerals containing gold, uranium, zinc, lead, arsenic, antimony, nickel, molybdenum, silver, and cobalt. Examples include galena, sphalerite, smithsonite, hemimorphite, zincite, willemite, hydrozincite, molybdenite, wulfenite, cobaltite, smaltite, erythrite, glaucodot, linnaeite, pentlandite, serpentine, saprolite, laterite, smectite, garnierite pentlandite, calaverite, krennerite, nagyagite, petzite and sylvanite.

The metal ore may be in a range of forms. For example, the metal ore may be provided in its native form, for example, in in situ operations, in pulverized form, in crushed form, for example as run-off mine (ROM) material, as granules, as a clay, or as a slurry.

### BRIEF DESCRIPTION OF THE DRAWINGS

FIG. 1 illustrates that contaminants can accumulate, temperature can drop, and overall leaching efficiency can decrease.

FIG. 2 demonstrates the control of the populations of microorganisms present in the microbial lixiviant is an effective and convenient way to maximize lixiviant functional lifespan and/or leaching efficiency

FIGS. 3 and 4 demonstrate that the copper leaching efficiency of a lixiviant comprising a rationally designed consortium of microorganisms belonging to different populations (iron oxidizers, sulfur oxidizers and organic carbon degraders.

FIG. 5 shows the modelled and a decline in the rate of copper extraction with increasing concentration of organic carbon when using Inoculant A and its recovery when replacing the inoculant with Inoculant B.

FIG. 6 irrigation commenced after one day of rest for agglomerants to stabilize and to reach target temperature and consisted of 1.7 liters of a synthetic (abiotic) raffinate being recirculated at a rate of 7 liters per m²/h.

FIG. 7 shows columns A, B, and C were additionally inoculated with moderately thermophilic populations of sulfur oxidizers (Acidothiobacillus caldus and Acidothiobacillus sulfuriphilus) and iron oxidizers (Sulfobacillus thermosulfidooxidans) on a continuous basis, referred to as ‘MC1’. Column D comprised a non-inoculated control.

### DETAILED DESCRIPTION

As explained above, the inventors have identified that through process control, the functional lifespan and/or leaching efficiency of the microbial lixiviant can be maximized. This is achieved through monitoring of one or parameters which are indicative of the function of the one or more populations of microorganisms.

In one embodiment, the method comprises making a temperature measurement. The temperature of the body of metal ore and/or the microbial lixiviant may be measured. The generation of heat by the microbial lixiviant is indicative of its function, specifically its metabolism of mineralic compounds in the ore such as recalcitrant sulfides. If a reduction of lixiviant and/or ore temperature is observed, this is indicative of a reduction in microbial function.

Measuring the temperature of the metal ore may be conducted using techniques known to those skilled in the art. For example, temperature probes may be used. In embodiments in which the body of metal ore is a heap, the temperature probe may be located on or within the heap. In embodiments in which the body of metal ore is provided in a tank, the temperature probe may be located within the tank.

In embodiments, the method comprises making an assessment of pyrite oxidation. Those skilled in the art will be familiar with techniques useful for assessing pyrite oxidation. For example, rock samples (e.g. via core samples) from the body of the metal ore which have been in contact with the microbial lixiviant can be analyzed to determine pyrite content. Additionally or alternatively, the composition of the microbial lixiviant itself and/or efflux from the body of ore can be analyzed. Pyrite is typically one of the most rapidly oxidized minerals and thus pyrite oxidation is a useful indicator of the function of iron oxidizing microorganisms within the microbial lixiviant. Additionally, the oxidation of pyrite releases substantial heat and thus controlling the rate of pyrite oxidation through selection of the microbial populations present in the microbial lixiviant permit the temperature of the body of metal ore to be controlled.

In some embodiments, if an increase in pyrite oxidation is measured in step iii) of the process of the invention, then, in step iv), the microbial lixiviant may be adjusted to adjust the number of iron oxidizing populations (e.g. through the addition of one or more iron oxidizing microbial populations to the lixiviant, or the addition of a second or subsequent microbial lixiviant/s comprising one or more iron oxidizing microbial populations) or to increase the number of iron oxidizing microbial populations present in the lixiviant.

In certain embodiments, the method may comprise assessing metabolite levels in the lixiviant. For a given microbial population, the production of specific metabolites is indicative of the metabolic function of that population. Thus, the levels of a specific metabolite may be determined. If a reduction in the level of a specific metabolite is observed, this may be indicative of a reduction in the metabolic function of one microorganism population in the microbial lixiviant.

Additionally or alternatively, the composition of metabolites may be assessed. If the profile of metabolites present in the lixiviant is altered, this is indicative of the abundance or metabolic function of microbial populations in the lixiviant shifting which could result in the microbial lixiviant metabolizing substrates other than the substrate of interest or the generation of contaminants which can damage the metabolic function of the microbial lixiviant.

The assessment of metabolite levels and/or composition may be achieved through obtaining a sample of metal ore contacted with the lixiviant, the lixiviant itself and/or efflux from the body of ore and assessing metabolite content using routine techniques. In embodiments in which the body of metal ore is a heap, a core sample from within the heap may be taken and the metabolite content assessed. Additionally or alternatively, the metabolite content of efflux from the heap may be assessed.

In certain embodiments, the method may comprise assessing nucleic acid profiles, for example in the lixiviant and/or in the body of metal ore. In embodiments, the nucleic acid may be DNA and/or RNA. The nucleic acid may be isolated.

In embodiments of the invention, the profiles of specific nucleic acid (e.g. DNA and/or RNA) sequences may be assessed. Thus, the levels of one or more specific sequences may be assessed. In embodiments, the levels of a specific sequence may be assessed, for example because the presence of that sequence is indicative of the function and/or prevalence of a specific strain of microorganism and/or a specific population of microorganisms. In certain embodiments, the levels of a number of different specific sequences may be assessed, for example to enable a determination to be made as to whether there are fluctuations in the identity and levels of nucleic acids which may be indicative of stressed and thus sub optimally performing populations.

In embodiments of the invention, the presence, absence or levels of specific sequences of nucleic acid may be assessed, for example the coded or transcribed 16S rRNA gene, coded or transcribed genes involved in microbial iron oxidation, coded or transcribed genes involved in microbial inorganic sulfur compound oxidation, coded or transcribed genes involved in the mediation of heavy metal toxicity in microorganisms. If specific sequences of nucleic acid are identified, or if levels of those sequences are altered, this is indicative of fluctuations in microbial community composition and/or functional capabilities.

The assessment of nucleic acid levels and/or the presence, absence and or levels of specific sequences may be achieved through obtaining a sample of metal ore, the microbial lixiviant and/or efflux from the body of ore and assessing nucleic acid content using routine techniques, for example DNA or RNA extraction, amplification via quantitative PCR, and/or sequencing, e.g., using Illumina sequencing technology. In embodiments in which the body of metal ore is a heap, a core sample from within the heap may be taken and the metabolite content assessed. Additionally or alternatively, the metabolite content of efflux from the heap may be assessed.

Additionally or alternatively the method may comprise determining the abundance and/or composition of the microbial populations present in the microbial lixiviant and/or the microbial diversity of the microbial lixiviant. The assessment of the abundance of the microbial populations present in the microbial lixiviant and/or the microbial diversity of the microbial lixiviant may be achieved through obtaining a sample of metal ore contacted with the lixiviant and assessing the microbial population using techniques including sequencing tools such as high throughput meta-genomic and transcriptomic or metatranscriptomic sequencing. Where the microbial diversity of the microbial lixiviant is assessed, this may comprise be presented using the Shannon index, the Chao1 index, or any other form of diversity index known to those skilled in the art. In embodiments in which the body of metal ore is a heap, a sample from within the heap and/or from its surface may be taken and the microbial composition/diversity assessed. Additionally or alternatively, the microbial composition/diversity in efflux from the heap may be assessed.

In certain embodiments, a pH measurement may be made. Variations in pH may be indicative of changes in the abundance of populations of microbes in the lixiviant. For example, an increase in the proportion of sulfur oxidizing microbes may result in the release of sulfuric acid causing a decrease in lixiviant pH.

pH may be measured using techniques known to those skilled in the art. In embodiments, pH may be measured by taking a sample of lixiviant and/or metal ore and assessing the pH. pH of metal ore in solid form may be assessed by firstly mixing the ore with a liquid medium, e.g. water, and then measuring pH. The sample of metal ore may be taken from within the body of metal ore and/or from the surface thereof. In embodiments (e.g. in which the body of metal ore is provided as a heap), the sample may be collected in efflux from the heap. Standard pH measurement apparatus may be employed, for example, a conventional pH meter.

In certain embodiments, if a decrease in pH is measured in step iii) of the process of the invention, then, in step iv), the microbial lixiviant may be adjusted to comprise one or more acid tolerant microbial populations (e.g. through the addition of one or more acid tolerant microbial populations to the lixiviant, or the addition of a second or subsequent microbial lixiviant/s comprising one or more acid tolerant microbial populations) or to increase the number of acid tolerant microbial populations present in the lixiviant.

In embodiments of the invention, organic carbon content levels may be measured. At initiation of a bioleaching process, organic carbon content is typically low, but can accumulate or increase over time due to inclusions of soil or clay in the ore body, introduction of foreign organic matter during ore processing, autotrophic microbial growth, and carryover from solvent extraction chemicals. Microorganisms which may be employed in bioleaching processes which effectively catalyse mineral dissolution may be sensitive to the inhibitory effects of organic carbon, for example at low pH. As the bioleaching process progresses, the populations present in the microbial lixiviant may be adjusted depending on the measured organic carbon.

In such embodiments, organic carbon content may be measured by taking a sample of lixiviant and/or metal ore and assessing the organic carbon content. The sample of metal ore may be taken from within the body of metal ore and/or from the surface thereof. In embodiments (e.g. in which the body of metal ore is provided as a heap), the sample may be collected in efflux from the heap.

In certain embodiments, if an increase in organic carbon content is measured in step iii) of the process of the invention, then, in step iv), the microbial lixiviant may be adjusted to comprise one or more microbial populations which degrade organic carbon (e.g. through the addition of one or more microbial populations which degrade organic carbon to the lixiviant, or the addition of a second or subsequent microbial lixiviant/s comprising one or more microbial populations which degrade organic carbon) or to increase the number of microbial populations which degrade organic carbon present in the lixiviant. Additionally or alternatively, the microbial lixiviant may be adjusted to comprise one or more microbial populations which are organic carbon tolerant, e.g. which maintain their function (such as iron oxidation and/or sulfur oxidation) in the presence of organic carbon. This could be achieved through the addition of one or more microbial populations which are organic carbon tolerant to the lixiviant, or the addition of a second or subsequent microbial lixiviant/s comprising one or more microbial populations which are organic carbon tolerant) or to increase the number of microbial populations which are organic carbon tolerant present in the lixiviant.

In some embodiments, the process may comprise measuring the levels of toxins, such as ore derived toxins or supplement-derived toxins (e.g. toxins deriving from supplements used in the process including catalysts, chloride and/or carbon black. Variations in the levels of such toxins may require adjustment of microbial populations which are more robust toward specific toxins (e.g. toxin tolerant population/s) and/or which are capable of metabolising the toxin or rendering it less toxic to the microbial lixiviant in some other way (e.g. toxin inactivating population/s), for example via sequestration or precipitation. As an example, metal ore bodies can contain fluoride at varying concentrations. Stacking of ore with increasing concentrations of fluoride can result in an increase in the concentration of fluoride in process fluids and in the heap. This increase in fluoride levels may negatively affect the function of microbial population. Thus, if an increase in fluoride levels is measured, the composition of the microbial lixiviant supplied to the heap may be adjusted to comprise alternative microbe populations with higher tolerance to fluoride.

Those skilled in the art will be familiar with compounds that may be released from metal ores in bioleaching processes which are toxic to microbes in lixiviant compositions. Examples of these include arsenic, halides including fluoride, lead and silver.

In such embodiments, ore-derived toxin content may be measured by taking a sample of lixiviant and/or metal ore and assessing the ore-derived toxin content. The sample of metal ore may be taken from within the body of metal ore and/or from the surface thereof. In embodiments (e.g. in which the body of metal ore is provided as a heap), the sample may be collected in efflux from the heap.

In certain embodiments, if an increase in toxin content is measured in step iii) of the process of the invention, then, in step iv), the microbial lixiviant may be adjusted to comprise one or more toxin tolerant and/or toxin inactivating microbial populations (e.g. through the addition of one or more toxin tolerant and/or toxin inactivating microbial populations to the lixiviant, or the addition of a second or subsequent microbial lixiviant/s comprising one or more toxin tolerant and/or toxin inactivating microbial populations) or to increase the number of toxin tolerant and/or toxin inactivating microbial populations present in the lixiviant.

In certain embodiments, an electrochemical parameter indicative of function of the microbial lixiviant may be measured. In embodiments, Oxidation Reduction Potential (ORP) may be assessed. For example, in leaching systems in which iron oxidizing microbes are comprised within the microbial lixiviant, this will impact the ratio of Fe³⁺:Fe²⁺ and thus increases in or the maintenance of ORP at elevated levels will be demonstrative of effective function of such lixiviants.

Copper primary minerals are often recalcitrant, but have been observed to be amenable to leaching at low ORP. As such, in some embodiments, ORP can be used as a measure of leaching efficiency. As ORP in sulfide leaching systems are determined by the ratio of Fe3+/Fe2+, modulation of populations of iron oxidizing microbes can be used to decrease ORP and thus increase leaching efficiency.

Those skilled in the art will be familiar with techniques and apparatus for measuring the ORP of lixiviant systems. In some embodiments, electrodes such as silver/silver chloride electrodes may be employed.

In certain embodiments, if a decrease in ORP is measured in step iii) of the process of the invention, then, in step iv), the microbial lixiviant may be adjusted to comprise one or more iron oxidizing microbial populations (e.g. through the addition of one or more iron oxidizing microbial populations to the lixiviant, or the addition of a second or subsequent microbial lixiviant/s comprising one or more iron oxidizing microbial populations) or to increase the number of iron oxidizing microbial populations present in the lixiviant.

In embodiments, one or more parameters indicative of function of the microbial lixiviant, such as those detailed above, may be measured. In certain embodiments, 2 or more such parameters may be measured. In embodiments, 3 or more such parameters may be measured. In certain embodiments, 4 or more such parameters may be measured. In some embodiments, 5 or more such parameters may be measured.

In the process of the invention, at least two measurements of a given parameter may be made. In such embodiments, one may be a baseline measurement, e.g. made at or around the time that the microbial lixiviant is contacted with the metal ore, with one or more subsequent measurements of that parameter being made following contacting of the metal ore with the microbial lixiviant. In such embodiments, a comparison may be made between the baseline measurement and the one or more subsequent measurements to determine whether there has been any change in the measured parameter.

In embodiments of the invention, steps iii) and iv) of the process of the invention may be repeated (optionally multiple times), i.e. such that multiple assessments of metabolic function of the microbial lixiviant are made and multiple interventions are carried out to ensure optimal leaching efficiency. In certain embodiments, one or more parameters indicative of function of the one or more populations of microorganisms may be continuously measured or may be intermittently measured. For the avoidance of doubt, it is not essential in the process of the invention that when a parameter is measured in step iii) of that process, an adjustment of one or more process variables must necessarily be made in response to the measured parameter; multiple measurements of the parameter may be made before an adjustment is made to the one or more process variables. In some embodiments, the parameter may be measured regularly over a period of time, for example the parameter may be measured hourly, every two hours, every three hours, every six hours, twice daily, once daily, twice weekly or once weekly. In certain embodiments, e.g. where measurement of the parameter is costly or challenging, yet still provides valuable information, the parameter may be measured monthly, bi-monthly, quarterly, every six months or annually. For the avoidance of doubt, where multiple parameters are measured in the process of the present invention, these do not have to be measured at the same time. For example, a first parameter (e.g. temperature or an electrochemical parameter such as ORP) may be measured daily, while a second parameter (e.g. 16S analysis) may be measured annually.

In embodiments, measurements of at least one parameter indicative of function of the one or more populations of microorganisms may be taken at different locations. For example, if the metal ore is provided as a heap, then measurements of one or more parameters indicative of function of the one or more populations of microorganisms may be taken at different locations within the heap.

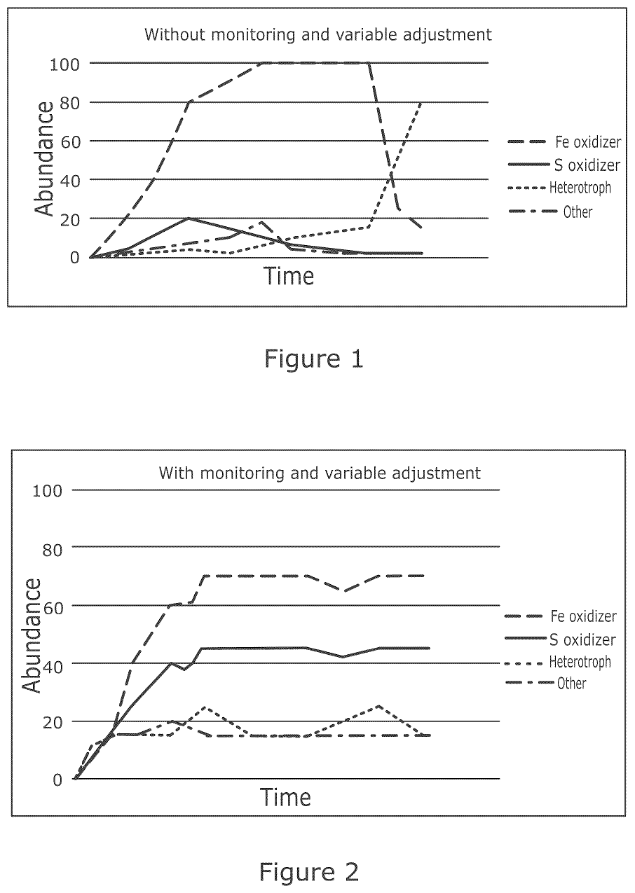

The inventors have found that the microbial diversity of a body of metal ore being subjected to a bioleaching process changes over time and while the total biomass present may be high, depending on the duration over which that biomass has been in contact with the body of metal ore, the biodiversity of the biomass can be depleted. As a consequence, contaminants can accumulate, temperature can drop, and overall leaching efficiency can decrease. This is graphically depicted in FIG. 1.

Thus, the inventors have recognized that the control of the populations of microorganisms present in the microbial lixiviant is an effective and convenient way to maximise lixiviant functional lifespan and/or leaching efficiency as depicted in FIG. 2. By doing so, lower levels of biomass can be supplied to bodies of metal ore such as heaps than in conventional processes where the aim was simply to maximise the biomass present in the heap on the assumption that this would maximise leaching function.

Thus, according to a further aspect of the present invention, there is provided a microbial lixiviant composition for use in a bioleaching process comprising a plurality of strains of microorganisms forming one or more populations of microorganisms selected from the following: iron oxidizers, sulfur oxidizers, organic carbon degraders and/or toxin inactivating microorganisms. Such compositions may be employed in the process of the present invention.

The microbial lixiviant may comprise bacteria, archaea, fungi, algae or other types of microorganism. Examples of genera of microorganisms which may be employed in the microbial lixiviants discussed herein include:
Iron-oxidizers: Acidocella, Acidimicrobium (e.g. Acidimicrobium ferrooxidans), Acidiphilium, Acidiplasma, Acidithiobacillus (e.g. Acidithiobacillus ferrivorans, Acidithiobacillus ferrooxidans, Acidithiobacillus ferrianus), Acidithiomicrobium, Acidithrix (e.g. Acidithrix ferrooxidans), Ferrithrix (e.g. Ferrithrix thermotolerans), Ferroplasma, Ferrovum (e.g. Ferrovum myxofaciens), Halomonas, Leptospirillum (e.g. Leptospirillum ferrooxidans, Leptospirillum ferriphilum), Metallosphaera (e.g. Metallosphaera sedula), Mycobacterium, Sulfobacillus (e.g. Sulfobacillus thermosulfidooxidans, Sulfobacillus thermotolerans), Sulfolobus, Sulfuracidifex (e.g. Sulfuracidifex metallicus—formerly Sulfolobus metallicus) and Thermogymnomonas.
Sulfur oxidizers: Acidianus (e.g. Acidianus sulfidivorans), Acidicaldus (e.g. Acidicaldus organivorans), Acidithiobacillus (e.g. Acidithiobacillus caldus, Acidithiobacillus sulfuriphilus, Acidithiobacillus albertensis, Acidithiobacillus ferrooxidans, Acidithiobacillus ferrianus), Ferviacidithiobacillus, Igneacidithiobacillus, Metallosphaera, Sulfobacillus (e.g. Sulfobacillus thermosulfidooxidans, Sulfobacillus thermotolerans), Sulfolobus (e.g. Sulfolobus acidocaldarius) and Sulfuracidifex (e.g. Sulfuracidifex metallicus, Sulfuracidifex tepidarus).
Organic carbon degraders: Acidicaldus (e.g. Acidicaldus organivorans), Acidianus, Acidocella, Acidimicrobium (e.g. Acidimicrobium ferrooxidans), Acidiphilium, Acidiplasma, Acidimicrobium (e.g. Acidimicrobium ferrooxidans), Acidithrix (e.g. Acidithrix ferrooxidans), Alicyclobacillus (e.g. Alicyclobacillus disulfoxidans), Ferrithrix (e.g. Ferrithrix thermotolerans), Ferroplasma, Ferrovum (e.g. Ferrovum myxofaciens), Metallosphaera, Picrophilus, Sulfobacillus (e.g. Sulfobacillus thermosulfidooxidans, Sulfobacillus thermotolerans), Sulfolobus and Thermoplasma.

A population that may be provided in the microbial lixiviant are toxin inactivating microorganisms. These organisms have the function of lowering the concentration of toxins in the microbial lixiviant and/or the body of metal ore such that the deleterious effects of toxins on the viability and/or function of microorganisms in the microbial lixiviant are lowered. In embodiments, the toxin inactivating microorganisms may have the function of precipitating toxins in non (or less) toxic forms, sequestering toxins and/or metabolizing toxins. Examples of toxins, the levels of which may be lowered by toxin inactivating microorganisms in the present invention include fluoride, arsenic, cobalt, zinc, lead, sulfate, and/or silver. In embodiments, the toxin may be chloride and/or nitrate and the toxin inactivating microorganism may only partially lower the levels of chloride and/or nitrate, and optionally not lower most or all of the chloride and/or nitrate present.

Additionally or alternatively, one, some or all of the strains of microorganisms present in the microbial lixiviant may be toxin tolerant.

In certain embodiments, one, some or all of the strains of microorganisms present in the microbial lixiviant may be capable of lowering and/or maintaining a low oxidation reduction potential (ORP) (for example about 500 to about 1000, about 500 to about 900 or about 600 to about 775 mV SHE) in the lixiviant.

In embodiments of the invention, the populations of microorganisms comprised in the microbial lixiviant and/or one or more of the microorganism populations comprised therein may be found together in nature. In alternative embodiments, one or more of the populations of microorganisms comprised in the microbial lixiviant may be artificially combined, i.e. the microbial lixiviant may comprise a rationally designed consortium of microorganisms.

In embodiments in which the microbial lixiviant comprises a rationally designed consortium of microorganisms, the process may comprise steps of providing a sample of the metal ore comprising microorganisms, identifying one or more populations of said microorganisms exhibiting target metabolic function, and isolating said one or more populations of microorganisms and providing said one or more populations of microorganisms in a microbial lixiviant which may then be employed in the process of the invention.

In such embodiments, the step of identifying one or more populations of microorganisms exhibiting target metabolic function may comprise culturing the sample of metal ore and identifying one or more populations of microorganisms which generate heat. The generation of heat may be measured using any technique known to those skilled in the art, for example microcalorimetry.

The microorganisms comprised in the sample of metal ore may be naturally comprised within that sample. Alternatively, the microorganisms may be manipulated, for example by removing certain populations of microorganisms naturally comprised in the sample of metal ore and/or adding populations of microorganisms to the sample of metal ore, e.g. to investigate their metabolic function.

In embodiments in which the step of identifying one or more populations of microorganisms exhibiting target metabolic function comprises culturing the sample of metal ore, this step may be conducted in the presence of one or more contaminants, for example contaminants commonly observed in mining process waters (e.g. released from metal ores during leaching processes, or comprised within lixiviants or other additives to aid and increase metal extraction and recovery), such as halides, arsenic, zinc, cobalt, thiourea, surfactants, nitrate, sulfate or combinations thereof.

As explained above, a critical step of the process of the present invention is adjustment of one or more process variables in response to a measured parameter. By carefully controlling the process variables, the leaching efficiency and/or functional lifespan of the microbial lixiviant can be maximized.

In embodiments, the process variable which is adjusted may be the composition of microorganisms present in the microbial lixiviant. For example, if the measured parameter is indicative of a shift in the microbial composition such that the relative abundance of one or more particular populations of microorganisms is altered to a sub-optimal level (e.g. if that population is depleted meaning that it will have reduced function, if that population is increased meaning that it will outcompete alternative populations of microorganisms which play a critical role in leaching for resources, or if all populations exhibit an unacceptable reduction in function) then the adjustment of the process variable may be the addition of a population of microorganisms (e.g. to increase the abundance of a population which is depleted and/or to increase the abundance of a population which lowers the levels of a toxin or organic carbon that may be responsible for a declining population) or the addition of a plurality of populations of microorganisms to lower the relative abundance of another population of microorganisms which is elevated). In embodiments, for example where measurement of the parameter is indicative of a widespread or complete loss of function or viability of the microbes present in the lixiviant, a total replacement of the microbial populations present in the microbial lixiviant may be contacted with the body of metal ore. In such embodiments the microbial populations may be the same or different to those originally contacted with the body of metal ore.

In embodiments in which the measurement of the parameter in step iii) of the process of the invention is indicative of a reduction in leaching effectiveness, the adjustment in step iv) may be to adjust the microbial composition of the lixiviant to revive or improve leaching effectiveness.

In some embodiments, the microbial lixiviant may be adjusted to comprise one or

more iron oxidizing microbial populations (e.g. through the addition of one or more iron oxidizing microbial populations to the lixiviant, or the addition of a second or subsequent microbial lixiviant/s comprising one or more iron oxidizing microbial populations) or to increase the number of iron oxidizing microbial populations present in the lixiviant.

In certain embodiments, the microbial lixiviant may be adjusted to comprise one or more sulfur oxidizing microbial populations (e.g. through the addition of one or more sulfur oxidizing microbial populations to the lixiviant, or the addition of a second or subsequent microbial lixiviant/s comprising one or more sulfur oxidizing microbial populations) or to increase the number of sulfur oxidizing microbial populations present in the lixiviant.

In some embodiments, the microbial lixiviant may be adjusted to comprise one or more organic carbon degrading microbial populations (e.g. through the addition of one or more organic carbon degrading microbial populations to the lixiviant, or the addition of a second or subsequent microbial lixiviant/s comprising one or more organic carbon degrading microbial populations) or to increase the number of organic carbon degrading microbial populations present in the lixiviant.

In embodiments of the invention, the microbial lixiviant may be adjusted to comprise one or more toxin inactivating microbial populations (e.g. through the addition of one or more toxin inactivating microbial populations to the lixiviant, or the addition of a second or subsequent microbial lixiviant/s comprising one or more toxin inactivating microbial populations) or to increase the number of toxin inactivating microbial populations present in the lixiviant.

In some embodiments of the invention, the microbial lixiviant may be adjusted to comprise one or more thermally resistant (such as moderately thermally resistant or highly thermally resistant, as defined herein) microbial populations (e.g. through the addition of one or more thermally resistant microbial populations to the lixiviant, or the addition of a second or subsequent microbial lixiviant/s comprising one or thermally resistant microbial populations) or to increase the number of thermally resistant microbial populations present in the lixiviant.

In certain embodiments of the invention, the microbial lixiviant may be adjusted to comprise one or more acid tolerant microbial populations (e.g. through the addition of one or more acid tolerant microbial populations to the lixiviant, or the addition of a second or subsequent microbial lixiviant/s comprising one or acid tolerant microbial populations) or to increase the number of acid tolerant microbial populations present in the lixiviant.

In certain embodiments of the invention, the microbial lixiviant may be adjusted to comprise one or more organic carbon tolerant microbial populations (e.g. through the addition of one or more organic carbon tolerant microbial populations to the lixiviant, or the addition of a second or subsequent microbial lixiviant/s comprising one or organic carbon tolerant microbial populations) or to increase the number of organic carbon tolerant microbial populations present in the lixiviant.

In certain embodiments of the invention, the microbial lixiviant may be adjusted to comprise one or more toxin tolerant microbial populations (e.g. through the addition of one or more toxin tolerant microbial populations to the lixiviant, or the addition of a second or subsequent microbial lixiviant/s comprising one or toxin tolerant microbial populations) or to increase the number of toxin tolerant microbial populations present in the lixiviant.

Thus, in embodiments, the process of the present invention comprises contacting the body of metal ore with a second microbial lixiviant, following step iii), comprising one or more populations of microorganisms wherein the microbial composition of the second microbial lixiviant differs from that of the microbial lixiviant contacted with the metal ore in step ii). The microbial composition of the second microbial lixiviant may comprise less populations of microorganisms as compared to the microbial lixiviant used in step ii). Alternatively, the microbial composition of the second microbial lixiviant may comprise more populations of microorganisms as compared to the microbial lixiviant used in step ii). Additionally or alternatively, the microbial composition of the second microbial lixiviant may comprise alternative microbial populations as compared to the microbial lixiviant used in step ii). In embodiments, the microbial composition of the second microbial lixiviant may comprise the same number of microbial populations, and optionally the same microbial populations, as the microbial lixiviant used in step ii) but in different proportions.

In embodiments of the invention, further compositions of microbial lixiviant (“subsequent microbial lixiviant/s”) may be supplied to the body of metal ore, in addition to the microbial lixiviant contacted with the body of metal ore in step ii) and the second microbial lixiviant. The microbial composition of the subsequent microbial lixiviant/s may comprise less populations of microorganisms as compared to the microbial lixiviant used in step ii) and/or the second microbial lixiviant. Alternatively, the microbial composition of the subsequent microbial lixiviant/s may comprise more populations of microorganisms as compared to the microbial lixiviant used in step ii) and/or the second microbial lixiviant. Additionally or alternatively, the microbial composition of the subsequent microbial lixiviant/s may comprise alternative microbial populations as compared to the microbial lixiviant used in step ii) and/or the second microbial lixiviant. In embodiments, the microbial composition of the subsequent microbial lixiviant may comprise the same number of microbial populations, and optionally the same microbial populations, as the microbial lixiviant used in step ii) and/or the second microbial lixiviant but in different proportions.

Where reference is made herein to properties of a microbial lixiviant, these may apply to the microbial lixiviant used in step ii) of the process of the present invention to the second microbial lixiviant and/or to the subsequent microbial lixiviant/s, if used.

In some embodiments, the one or more microbial populations comprised within the microbial lixiviant may be thermally resistant, e.g. moderately thermally resistant or highly thermally resistant as described herein. For example, in certain embodiments, if an increase in temperature is measured in step iii) of the process of the invention, then, in step iv), the microbial lixiviant may be adjusted to comprise one or more thermally resistant microbial populations (e.g. through the addition of one or more thermally resistant microbial populations to the lixiviant, or the addition of a second or subsequent microbial lixiviant/s comprising one or more thermally resistant microbial populations) or to increase the number of thermophilic microbial populations present in the lixiviant.

Additionally or alternatively, other methods of adjusting the composition of the microorganisms present in the microbial lixiviant may be employed, for example the application of a phage specific to one of the populations of microorganisms or a subset of microorganisms in such a population, the application of an antimicrobial specific to one of the populations of microorganisms or a subset of microorganisms in such a population and/or the application of one or more nutrients which selectively promotes the growth of one or more populations of microorganisms.

Additional process variables which may be adjusted in the methods of the present invention include the rate of application of the microbial lixiviant to the body of metal ore, the temperature at which the metal ore is maintained and/or agitation or other mechanical disruption of the metal ore. Those skilled in the art will recognize that the control of certain process variables may be more readily achievable depending on the way in which the process is conducted. For example, if the body of metal ore is provided in a tank, then this may make control of the temperature at which the process is operated more straightforward. However, all combinations of process variables and types of leaching process are encompassed by the present invention.

Conveniently, the microbial lixiviants discussed herein may be cultured and/or prepared at the site at which those lixiviants will be contacted with the body of metal ore. For example, bioreactor apparatus optionally comprising one or more bioreactors, may be provided in the vicinity of the body of metal ore (e.g. within about 10 km, about 5 km, about 2 km, about 1 km, about 500 m, about 200 m, about 100 m, about 50 m, about 20 m or about 10 m). A biomass may be recovered from the bioreactor apparatus. The biomass may be contacted directly with the body of metal ore (in which case the biomass will be the microbial lixiviant (or second microbial lixiviant and/or subsequent microbial lixiviant/s, if used)). Alternatively, the biomass may be fed into a lixiviant stream which is then contacted with the body of metal ore as the microbial lixiviant (or second microbial lixiviant and/or subsequent microbial lixiviant/s, if used). In embodiments, the biomass recovered from the bioreactor apparatus may comprise about 10⁶ to about 10¹³ cells/L of viable microorganisms. In certain embodiments, the biomass recovered from the bioreactor apparatus may comprise about 10⁷ or 10⁸ to about 10¹² cells/L of viable microorganisms.

In some embodiments, the bioreactor apparatus may be transportable. In embodiments, the population/s comprised within the microbial lixiviant are cultured and/or prepared at the site at which those lixiviants will be contacted with the body of metal ore. For example, bioreactor apparatus optionally comprising one or more bioreactors, may be provided in the vicinity of the body of metal ore (e.g. within about 10 km, about 5 km, about 2 km, about 1 km, about 500 m, about 200 m, about 100 m, about 50 m, about 20 m or about 10 m).

As used herein, the term ‘transportable’ is used to mean that the bioreactor apparatus can be shipped essentially operationally intact from one location to another. Preferably the bioreactor apparatus is containerized in operationally intact form. A suitable form of containerization may be provided in the form of a container substantially similar in size and shape to a standard shipping container. By “operationally intact” is meant that the bioreactor apparatus may be commissioned at a first location such that at least one microbial population (or component strain or strains thereof) is fermented within a bioreactor comprised within the bioreactor apparatus and subsequently transported to a second location (optionally the site of the body of metal ore), where the microbial population (or component strain or strains thereof) continues to be nurtured within the bioreactor.

The bioreactor apparatus is provided with one or more bioreactors for fermenting microorganisms, including means for providing microorganisms in the bioreactor/s with one or more nutrients and/or adjuvants for encouraging or selectively encouraging the growth of the at least one microbial population (or component strain or strains thereof) in the bioreactor/s, and with means for maintaining fermentation conditions, e.g. of temperature, pressure and/or chemical environment (e.g. salinity) within the bioreactor/s conducive to the fermentation of the microbial population (or component strain or strains thereof).

The bioreactor apparatus may therefore be provided in the form of a containerized and transportable unit which may be shipped to the site of the body of metal ore and provided with means for supplying the microbial population (or component strain or strains thereof) from the bioreactor into or into the vicinity of the body of metal ore.

In some embodiments of the present invention the transportable bioreactor apparatus may be accompanied by—or have incorporated within it—a nutrient tank. The nutrient tank may contain any required nutrients or adjuvants for the growth of the microbial population (or component strain or strains thereof. In a preferred embodiment of the present invention, the nutrient tank may also be contained within the transportable bioreactor apparatus, providing a single compact unit that is easier to transport to and from metal ore bodies.

In embodiments, the bioreactor apparatus may comprise a plurality of bioreactors. Alternatively, the bioreactor apparatus may comprise a single bioreactor and multiple bioreactor apparatus may be employed.

In embodiments, a first bioreactor may be used to ferment a first microbial population and a second bioreactor may be used to ferment a second microbial population.

In certain embodiments, the bioreactor apparatus may be used to produce a plurality of microbial populations which may be comprised within the microbial lixiviant (or second microbial lixiviant and/or subsequent microbial lixiviant/s, if used)).

Thus, according to a further aspect, there is provided a kit comprising a bioreactor apparatus and/or a microbial lixiviant composition as described herein. The kit may additionally comprise instructions for using the kit to perform the process as described herein. In some embodiments, the instructions require the bioreactor apparatus to be situated within about 1 km, about 500 m, about 200 m, about 100 m, about 50 m, about 20 m or about 10 m of the body of metal ore.

In embodiments of the invention, the process may comprise the step of providing the microbial lixiviant with a nutrient composition. This may be combined within the microbial lixiviant prior to it being contacted with the body of metal ore and/or be contacted with the microbial lixiviant after it the microbial lixiviant is contacted with the metal ore. Such nutrient composition may comprise microbial energy sources, for example raffinate.

In embodiments of the invention, the microbial lixiviant may comprise one or more components employed in conventional leach lixiviants, for example an acid (e.g. sulfuric acid) and/or a source of ferric iron (for example a ferrous salt such as ferrous sulfate which can be converted microbially into ferric iron, or a ferric salt such as ferric sulfate).

In certain embodiments of the invention, the microbial lixiviant is acidic. In such embodiments, the pH may be about 0 to about 5, about 1 to about 4, about 1 to about 3 or about 2 to about 4.

In certain embodiments, the microbial lixiviant may comprise an nutrient source, e.g. ammonium (e.g. ammonium sulfate), phosphate (e.g. potassium phosphate), carbonate (e.g., sodium carbonate), and/or nitrate (e.g. calcium nitrate).

In an embodiment, the microbial lixiviants discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may comprise microorganisms in an amount of at least about 10 cells/mL, at least about 10³ cells/mL, at least about 10⁴ cells/mL, at least about 10⁵ cells/mL, at least about 10⁶ cells/mL, at least about 10⁷ cells/mL or at least about 10⁸ cells/mL.

In alternative embodiments, the microbial lixiviants discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may comprise microorganisms in an amount of about 10¹⁰ cells/mL or lower, about 10⁸ cells/mL or lower, about 10⁷ cells/mL or lower, about 10⁶ cells/mL or lower, about 10⁵ cells/mL or lower, or about 10⁴ cells/mL or lower.

An advantage of the present invention is that, unlike conventional bioleaching processes, the monitoring of microbial function and/or viability through the measurement of parameter/s permits a comprehensive understanding of the abundance and/or function of specific microbial populations. This in turn enables effective leaching efficiency to be maintained through the periodic provision of smaller numbers of microorganisms to the body of metal ore, in contrast to the prior art approach of simply supplying ore with vast volumes of microbes in the hope of this translating into leaching efficiency. Thus, in embodiments of the invention, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise microorganisms in an amount from about 10² cells/mL to about 10⁸ cells/ml. In embodiments, the microbial lixiviant discussed herein may comprise microorganisms in an amount from about 10² cells/mL to about 10⁷ cells/mL. In embodiments the microbial lixiviant discussed herein may comprise microorganisms in an amount of from about 10³ cells/mL to about 10⁶ cells/mL.

In some embodiments of the invention, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may consist of essentially 2 or more, 3 or more, 4 or more, 5 or more, 6 or more, 7 or more, 8 or more, 9 or more, 10 or more, 11 or more, 12 or more, 13 or more, 14 or more, 15 or more or 16 or more strains of microorganisms. Additionally or alternatively, the microbial lixiviant discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may consist essentially of 50 or fewer, 40 or fewer, 30 or fewer, or 20 or fewer, 10 or fewer, 8 or fewer, 7 or fewer, 6 or fewer or 5 or fewer strains of microorganism. In certain embodiments, the microbial lixiviant discussed herein may consist essentially of 2 to 50 strains of microorganisms. In an embodiment the microbial lixiviant discussed herein may consist essentially of 2 to 30 strains of microorganisms. In a further embodiment, the microbial lixiviant discussed herein may consist of essentially 2 to 20 strains of microorganisms. The microbial lixiviant discussed herein may consist essentially of 2 to 10 strains of microorganisms. In a specific embodiment, the microbial lixiviant discussed herein may consist essentially of 2 to 8 strains of microorganism. In further embodiments, the microbial lixiviant discussed herein may consist essentially of 2 to 6 strains of microorganism or 2 to 5 strains of microorganism.

In embodiments of the invention, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁴ cells/mL.

In embodiments of the invention, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁵ cells/mL.

In embodiments of the invention, the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁶ cells/mL.

As used herein, the term “population” is used to describe a group of strains of microorganisms exhibiting common function. Examples of populations of microorganisms which may be present in microbial lixiviants as disclosed herein include iron oxidizers, sulfur oxidizers, organic carbon degraders and/or toxin inactivating microorganisms. For the avoidance of doubt, a single strain having varied functionality such that it could be considered to belong to more than one of the populations discussed herein, that strain shall nevertheless be considered to belong to only a single population. For example, if a composition comprises a single strain which has iron oxidizing functionality and sulfur oxidizing functionality, then for the purposes of the present disclosure, that composition will be considered to comprise one population, and not two populations.

In addition to the common function of the population/s comprised in the microbial lixiviants, microbial strains present in a population may additionally exhibit tolerance to leaching conditions. For the avoidance of doubt, as used herein, the term ‘tolerance’ means that the strains present in a population will exhibit their common function (e.g. they will function as iron oxidizers, sulfur oxidizers, organic carbon degraders and/or toxin inactivators) when exposed to different leaching conditions.

In some embodiments, one, some or all of the populations present in the microbial lixiviant may be thermally tolerant, for example, they may exhibit their common function at a temperature of greater than about 5° C., greater than about 10° C., greater than about 15° C. or greater than about 20° C. In some embodiments, one, some or all of the populations present in the microbial lixiviant may be moderately thermally tolerant, e.g. they may exhibit their common function at a temperature in the range of about 10° C. to about 60° C., about 15° C. to about 55° C. or about 20° C. to about 50° C. In certain embodiments, one, some or all of the populations present in the microbial lixiviant may be highly thermally tolerant, e.g. they may exhibit their common function at a temperature in the range of about 30° C. to about 100° C., about 40° C. to about 90° C. or about 50° C. to about 80° C. Microorganisms comprised in a thermally tolerant population may be thermophilic or mesophilic.

In some embodiments, one, some or all of the populations present in the microbial lixiviant may be acid tolerant, for example, they may exhibit their common function at a pH of about 7 or lower, about 6 or lower, about 5 or lower, about 4 or lower, about 3 or lower, or about 2 or lower. In some embodiments, a population may be moderately acid tolerant, for example, it may exhibit its common function at a pH of about pH 7 or lower to about 3 or higher or about pH 6 or lower or about 3.5 or higher. In certain embodiments, a population may be highly acid tolerant, for example, it may exhibit its common function at a pH of about pH 5 or lower to about 1 or higher or about pH 4 or lower or about 2 or higher. Microorganisms comprised in an acid tolerant population may be acidophilic.

In some embodiments, one, some or all of the populations present in the microbial lixiviant may be organic carbon tolerant, for example, they may exhibit their common function in the presence (optionally in greater than de minimis levels) of organic carbon which may be generated during the leaching process, be introduced during copper recovery processes, originate from contaminants and/or be added as supplements. In certain embodiments, the population/s maintain their common function in lixiviants comprising about 50 ppm or more, about 100 ppm or more, about 200 ppm or more, about 500 ppm or more, about 1000 ppm or more, about 2000 ppm or more or about 5000 ppm or more organic carbon. Such populations may or may not also be organic carbon degraders.

In some embodiments, one, some or all of the populations present in the microbial lixiviant may be toxin tolerant, for example, they may exhibit their common function in the presence of toxins present (optionally in greater than de minimis levels) in the microbial lixiviant. As used herein, the term ‘toxins’ encompasses compounds which adversely affect the viability and biological function of microbes and which may be present as components of the microbial lixiviant (e.g. supplements such as carbon black, catalysts) and/or be generated during the leaching process, such as ore-derived toxins e.g. halide ions such as chloride and/or fluoride, metals such as arsenic, zinc, cobalt, lead, silver, or other compounds such as thiourea, surfactants, nitrate, sulfate or combinations thereof. Such populations may or may not also be toxin inactivating microorganisms. Thus, in some embodiments, a microbial lixiviant employed in the present invention may comprise a population which is iron oxidizing and thermally tolerant. In certain embodiments, a microbial lixiviant employed in the present invention may comprise a population which is iron oxidizing and acid tolerant. In embodiments, a microbial lixiviant employed in the present invention may comprise a population which is iron oxidizing and organic carbon tolerant. In some embodiments, a microbial lixiviant employed in the present invention may comprise a population which is iron oxidizing and toxin tolerant.

In some embodiments, a microbial lixiviant employed in the present invention may comprise a population which is sulfur oxidizing and thermally tolerant. In certain embodiments, a microbial lixiviant employed in the present invention may comprise a population which is sulfur oxidizing and acid tolerant. In embodiments, a microbial lixiviant employed in the present invention may comprise a population which is sulfur oxidizing and organic carbon tolerant. In some embodiments, a microbial lixiviant employed in the present invention may comprise a population which is sulfur oxidizing and toxin tolerant.

In some embodiments, a microbial lixiviant employed in the present invention may comprise a population which is organic carbon degrading and thermally tolerant. In certain embodiments, a microbial lixiviant employed in the present invention may comprise a population which is organic carbon degrading and acid tolerant. In embodiments, a microbial lixiviant employed in the present invention may comprise a population which is organic carbon degrading and organic carbon tolerant. In some embodiments, a microbial lixiviant employed in the present invention may comprise a population which is organic carbon degrading and toxin tolerant.

In some embodiments, a microbial lixiviant employed in the present invention may comprise a population which is toxin inactivating and thermally tolerant. In certain embodiments, a microbial lixiviant employed in the present invention may comprise a population which is toxin inactivating and acid tolerant. In embodiments, a microbial lixiviant employed in the present invention may comprise a population which is toxin inactivating and organic carbon tolerant. In some embodiments, a microbial lixiviant employed in the present invention may comprise a population which is toxin inactivating and toxin tolerant.

The compositions disclosed herein may comprise one or more populations, two or more populations, three or more populations or four or more populations. The one or more populations, two or more populations, three or more populations or four or more populations may be selected from iron oxidizers, sulfur oxidizers, organic carbon degraders and/or toxin inactivating microorganisms. In certain embodiments, the compositions disclosed herein may comprise 1 to 4, 1 to 3, 1 to 2, 2 to 3, 2 to 4 or 3 to 4 of said populations.

In embodiments, the microorganisms within a population may all belong to the same species, the same genus, the same family, the same order, the same class, the same phylum, the same kingdom or the same domain. Alternatively, the microorganisms within a population may belong to 1 or more, 2 or more, 3 or more, 4 or more, or 5 or more, genera of microorganism. In embodiments, the microorganisms within a population may belong to 1 or more, 2 or more, 3 or more, 4 or more, or 5 or more species of microorganism.

As used herein, to describe the bacterial population comprised within the composition of the invention, the term ‘consisting essentially of” and ‘consisting of’ is used to characterize the composition as excluding additional bacterial strains or species, or comprising only de minimis or biologically irrelevant amounts of other bacterial strains or species.

In some embodiments of the invention, a population of microorganisms may consist of essentially 1 or more, 2 or more, 3 or more, 4 or more, 5 or more, 6 or more, 7 or more, 8 or more, 9 or more or 10 or more strains of microorganisms. Additionally or alternatively, a population of microorganisms may consist essentially of 50 or fewer, 40 or fewer, 30 or fewer, 20 or fewer, 10 or fewer, 8 or fewer, 6 or fewer, 5 or fewer, 4 or fewer, 3 or fewer or 2 or fewer strains of microorganism. In certain embodiments, a microorganism population may consist essentially of 1 to 50 strains of microorganisms. In an embodiment, a microorganism population may consist essentially of 1 to 30 strains of microorganisms. In a further embodiment, a microorganism population may consist of essentially 1 to 20 strains of microorganisms. A microorganism population may consist essentially of 1 to 10 strains of microorganisms. In a specific embodiment, a microorganism population may consist essentially of 1 to 5 strains of microorganism. In further embodiments, a microorganism population may consist essentially of 1 to 3 strains of microorganism.

In an embodiment, one, some or all of the populations comprised in the microbial lixiviants discussed herein, e.g. the compositions defined herein and/or the microbial lixiviant/s supplied to the body of metal ore (for example, the lixiviant contacted with the body of metal ore in step ii), the second microbial lixiviant and/or the subsequent microbial lixiviant/s) may comprise microorganisms in an amount of at least about 10² cells/mL, at least about 10³ cells/mL, at least about 10⁴ cells/mL, at least about 10⁵ cells/mL, at least about 10⁶ cells/mL, at least about 10⁷ cells/mL or at least about 10⁸ cells/mL.

In embodiments of the invention, one, some or all of the populations comprised in the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁴ cells/mL.

In embodiments of the invention, one, some or all of the populations comprised in the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁵ cells/mL.

In embodiments of the invention, one, some or all of the populations comprised in the microbial lixiviant discussed herein, e.g. the compositions defined herein and/or the microbial lixiviant/s supplied to the body of metal (for example, the lixiviant contacted with the body of metal ore in step ii), the second microbial lixiviant and/or the subsequent microbial lixiviant/s) ore may comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more or 16 or more, or 2 to 10, 2 to 8, 2 to 6, 2 to 5 or 2 to 4 strains of microorganisms having an abundance of at least 10⁶ cells/mL.

In embodiments of the invention, the microbial lixiviant discussed herein may be free of genetically modified microorganisms. In alternative embodiments, the microbial lixiviant discussed herein may principally or exclusively comprise microorganisms which are not genetically modified microorganisms. In such embodiments, at least 50%, at least 60%, at least 70%, at least 80%, at least 90%, at least 95%, at least 98%, at least 99% or at least 99.5% of the microorganisms present in the microbial lixiviant are not genetically modified organisms.

In other embodiments of the invention, the microorganisms present in the microbial lixiviant may principally or exclusively comprise organisms which are genetically modified. In such embodiments, at least 50%, at least 60%, at least 70%, at least 80%, at least 90%, at least 95%, at least 98%, at least 99% or at least 99.5% of the microorganisms present in the microbial lixiviant are genetically modified organisms.

In an embodiment, microbial lixiviant may be continuously supplied to the body of metal ore. As used herein “continuous” or “continuously” is not to be interpreted in a strictly literal sense but also encompasses situations in which the supply of microbial lixiviant may be temporarily paused, e.g. to permit the change or replacement of apparatus or sources of lixiviant to be contacted with the body of metal ore. The continuous supply of microbial lixivant to a body of metal ore may mean that the body of metal ore is continuously supplemented with microbial lixiviant comprising a functioning and effective population or populations of microorganisms for the duration of the bioleaching process.

Continuously supplying a microbial lixiviant comprising functionally diverse microorganisms can buffer chemical imbalances and prevent the accumulation of contaminants such as sulfur or organic carbon rapidly and locally before they become a factor that impacts leaching efficiency. In certain embodiments, the microbial composition and/or rate of supply of microbial lixiviant to the body of metal ore may be controlled such that the adjustment of the microbial populations in the microbial lixiviant and/or the body of metal ore is gradual. In normal operation of the process, such gradual control of the microbial composition is preferable as this permits leaching activity to continue without interruptions caused by populations which are either not present or which are present but at low levels being added and needing to establish an effective population. However, in other situations, for example, when the measured parameters are indicative of a rapid and/or significant loss in microbial viability and/or function, then the one or more microbial populations may need to be contacted with the body of metal ore, via the lixiviant, in high volumes and/or at high flow rates.

In an embodiment, microbial lixiviant is added to the body of metal ore at a rate of about 10 L/h per m² of surface of the body of metal ore.

The process of the present invention allows for the efficient recovery of metals from metal ores. The metal of interest may be present in the ore at a level of 5% or less by weight. However, in embodiments, the metal of interest may be present in the ore at lower levels, for example at a level of 4% or less, 3% or less, 2% or less, or 1% or less.

In certain embodiments, the metal of interest may be present in the ore at a level of about 5000 ppm or less, at a level of about 2000 ppm or less, at a level of about 1000 ppm or less, about 500 ppm or less, about 200 ppm or less, about 100 ppm or less or about 50 ppm or less. Additionally or alternatively, the metal of interest may be present in the ore at a level of at least about 1 ppm, at least about 2 ppm, at least about 5 ppm, at least about 10 ppm, at least about 20 ppm, at least about 50 ppm, or at least about 100 ppm.

In some embodiments of the present invention, the metal ore will be contacted with microbial lixiviant about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. In some embodiments of the invention, the metal ore will be contacted with microbial lixiviant about every 1-30, 1-25, 1-20, 1-15, 1-10, or 1-5 days.

Following contact of the body of metal ore with the microbial lixiviant (and second microbial lixiviant and/or subsequent microbial lixiviant/s, if used), metal will be released from the metal ore and thus, in embodiments of the invention, the process further comprises collecting a stream from the body of metal ore comprising the metal. This may be achieved through using techniques familiar to one skilled in the art.

In embodiments, a lixiviant efflux stream may be collected from the body of metal ore and this may be recycled to the body of metal ore. Prior to being recycled, the microbial composition, diversity and/or metabolic function of the microbial lixiviant may be assessed. Additionally or alternatively, the lixiviant efflux stream may be treated, e.g. by the addition of microorganisms belonging to one or more microorganism populations.

In embodiments of the invention, the process of the invention comprises the step of determining the pyrite composition of the body of metal ore and subsequently making one or more assessments of pyrite oxidation. In such embodiments, one or more assessments of the temperature of the body of metal ore may also be made. In such embodiments, if the pyrite oxidation exceeds a predetermined level, then the microbial composition of the microbial lixiviant may be adjusted, for example, to reduce the size of, or remove the population of iron oxidizing microorganisms. Alternatively, if the pyrite oxidation falls below a predetermined level, then the microbial composition of the microbial lixiviant may be adjusted, for example to increase the size of the population of iron oxidizing microorganisms. Additionally or alternatively, pyrite may be added to the body of metal ore.

The invention will now be illustrated by the examples which follow.

### EXAMPLES

### Example 1

A 1:1 mix of copper concentrate comprising mainly chalcopyrite with a 26.8% copper content and 27.4% iron content, and raw copper ore with 0.6% copper, 1% iron, was used.

1 g of the ore mix and 100 ml of leaching solution (3.0 g/L (NH₄)₂SO₄, 0.5 g/L K₂HPO₄, 0.5 g/L MgSO₄×7H₂O, 0.1 g/L KCl and 0.1 g/L Ca(NO₃)₂) adjusted to a pH of 1.8 with sulfuric acid were mixed and poured into a 250 ml stirred flask. This mixture was stirred at 35° C. A biomass comprising 10⁶ cells/mL was added to the mixture. The biomass comprised the following strains: Acidithiobacillus thiooxidans (sulfur oxidizer), Ferroplasma acidophilum (iron oxidizer, organic carbon degrader), Leptospirillum ferrooxidans (iron oxidizer), Sulfobacillus thermosulfidooxidans (iron oxidizer, sulfur oxidizer, organic carbon degrader).

As a comparative example, an identical ore/lixiviant slurry was prepared but was not inoculated with microorganisms and was kept sterile.

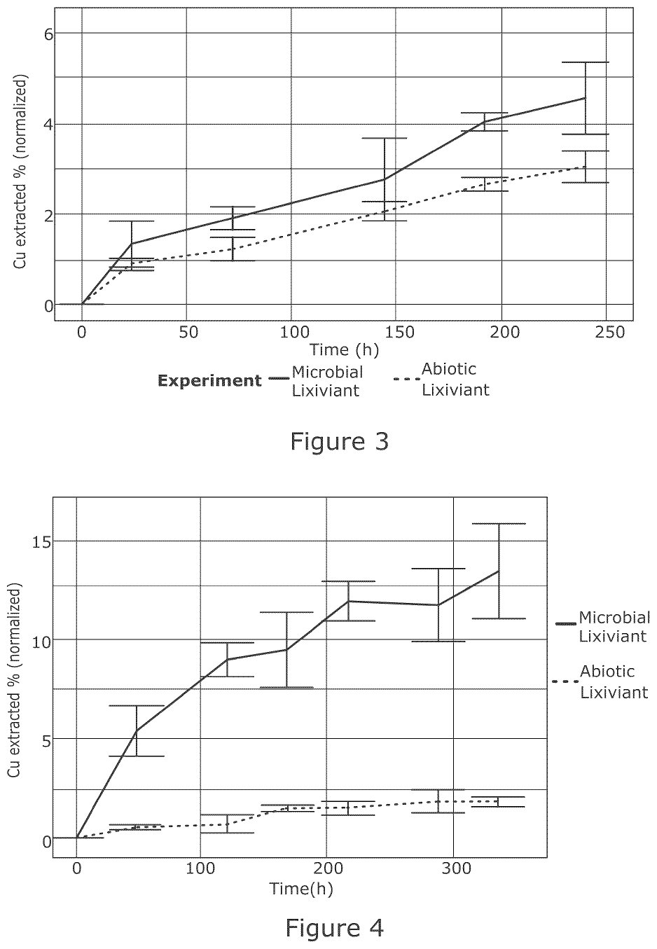

The copper recovered was measured using atomic absorption spectrometry (MS) and the results are shown in FIG. 3.

The experiment was repeated using identical conditions except that a different biomass was used comprising 10⁶ cells/mL of the following strains: Acidithiobacillus ferrooxidans (sulfur oxidizer, iron oxidizer) and Leptospirillum ferriphilum (iron oxidizer).

The copper recovered was measured using atomic absorption spectrometry (MS) and the results are shown in FIG. 4.

The data shown in FIGS. 3 and 4 demonstrate that the copper leaching efficiency of a lixiviant comprising a rationally designed consortium of microorganisms belonging to different populations (iron oxidizers, sulfur oxidizers and organic carbon degraders in the case of the results shown in FIG. 3 and iron oxidizers and sulfur oxidizers in the case of the results shown in FIG. 4) are greater than a conventional abiotic lixiviant.

### Example 2

In a modelled example, two 50 kg samples of copper ore having an average diameter of 0.5 to 25 mm and representative in composition to ore commonly found in industrial mining operations in Arizona was added to a leach column and irrigated with a synthetic raffinate at a flow of 10 liters per m² per hour. The synthetic raffinate was continuously supplemented with a mix of iron and sulfur-oxidizing, mesophilic, autotrophic and organic carbon-sensitive, acidophilic bacteria such as typically found in metal sulfide heap leaching to a concentration of 10⁴ cells/ml in the raffinate (Inoculant A). Copper contents in solution at the bottom of the leach column were measured using atomic absorption spectrometry (MS), extracting filtered samples in a micro-porous material (5 μm pore diameter).

Simulated organic carbon content in the raffinate was gradually increased by addition of YE until a concentration of 0.02% was reached on day 40, at which point raffinate inoculation of one column was switched to a mix of iron and sulfur-oxidizing, mesophilic, organic carbon tolerant and degrading, acidophilic bacteria at the same concentration (Inoculant B) while for the other column the original inoculant composition was retained. Incubation continued until day 100.

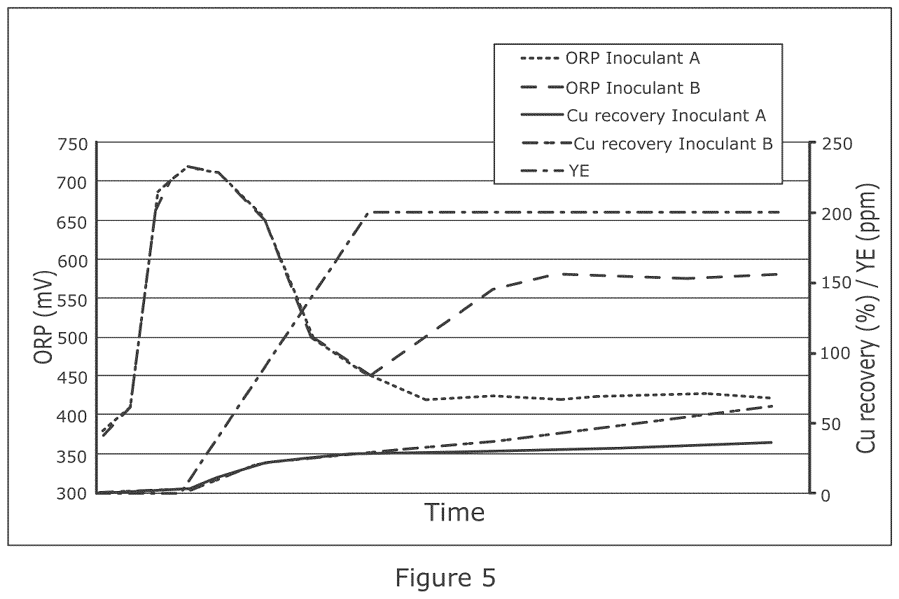

The modelled data is shown in FIG. 5 and shows a decline in the rate of copper extraction with increasing concentration of organic carbon when using Inoculant A and its recovery when replacing the inoculant with Inoculant B.

This modelled data demonstrates that, by monitoring variables in a body of metal ore such as organic carbon content, the microbial composition in the lixiviant added to that body can be tailored to maximize leaching efficiency.

### Example 3

1 kg of a low grade copper sulfide ore (copper content 3064 ppm) was crushed to an average particle size of 6.5 mm, agglomerated using a sulfuric acid solution, and packed into four 50 mm diameter leach columns (A to D) to a bed height of approximately 400 mm.

The columns were placed in an incubator at a temperature of 45° C. and forcibly aerated from the bottom, receiving humidified air at a rate of 50 ml/min.

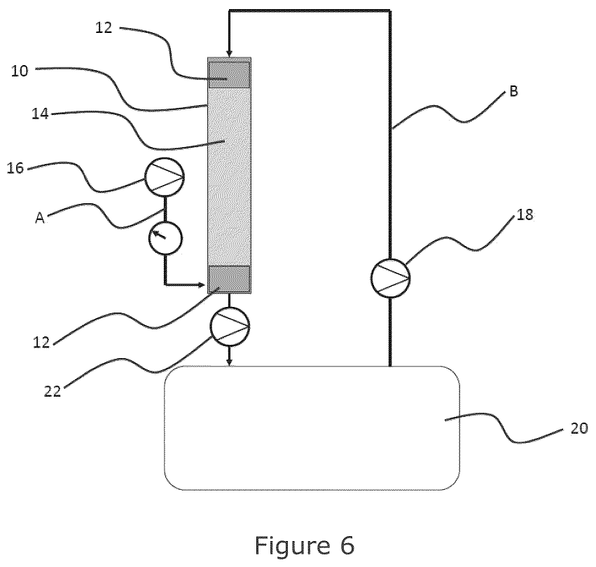

Irrigation commenced after one day of rest for agglomerants to stabilize and to reach target temperature and consisted of 1.7 litres of a synthetic (abiotic) raffinate being recirculated at a rate of 7 litres per m²/h. The set up of the columns is depicted in FIG. 6. Specifically, column 10 comprises sand beds 12 at its upper and lower ends to allow proper flow of process fluids. The central region of the column 14 comprises the agglomerated ore. Air pump 16 provides air into the column 10 via line A at a rate of 0.05 litres per minute. Pump 18 pumps synthetic raffinate from the reservoir 20 via line B into the column 10 at a rate of 14 ml per hour. Pregnant liquor solution obtained from column 10 is pumped by pump 22 back into the reservoir 20.

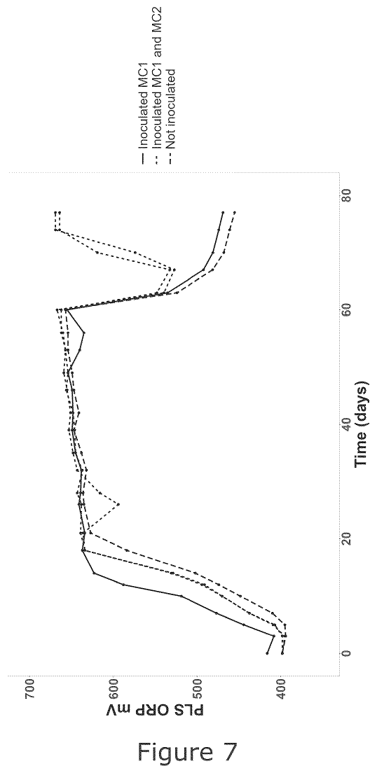

Columns A, B, and C were additionally inoculated with moderately thermophilic populations of sulfur oxidizers (Acidothiobacillus caldus and Acidothiobacillus sulfuriphilus) and iron oxidizers (Sulfobacillus thermosulfidooxidans) on a continuous basis, referred to as ‘MC1’ in FIG. 7. Column D comprised a non-inoculated control.

ORP in the recirculating leach liquor versus a Ag/AgCl electrode was measured daily for 60 days, confirming oxidative leaching of the sulfide ore. On day 60, a temperature change was artificially induced by adjusting the incubator's target temperature, and temperature was increased to 65° C. over 4 days.

As shown in FIG. 7, a sudden and severe decrease in ORP indicated a crash of the microbial leach catalysis and a disruption of the oxidative leaching of the sulfide ore on day 63.

Leaching with an alternative microbial composition (referred to as MC2 in FIG. 7) comprising highly thermally tolerant populations of sulfur oxidisers (Acidianus sulfidivorans) and iron oxidizers (Sulfuracidifex metallicus) commenced on the same day to reestablish leaching in Columns A and B. No alterations were made to the liquors present in Columns C and D.

An effect of this substitution of microbial populations on ORP was observed on day 67. On day 74, optimal leaching conditions were reestablished in Column A and B while leaching in non-adjusted columns C and D remained disrupted.

This data demonstrates that the repeated monitoring of a parameter (in this case, ORP) is a useful indicator of leaching effectiveness. Further, the data demonstrates that, if a change is observed which adversely impacts leaching efficiency (in this case, an increase in temperature), leaching effectiveness can be revived or maintained through the careful control of the microbial population/s comprised in lixiviant.

What is claimed is:

1. A process comprising:
i) providing a body of metal ore;
ii) contacting the body of metal ore with a microbial lixiviant comprising one or more populations of microorganisms;
iii) measuring at least one parameter indicative of function of the one or more populations of microorganisms; and
iv) adjusting one or more process variables in response to the measured parameter.

2. The process of claim 1 wherein the body of metal ore is a heap, is provided in a tank or is contacted with the microbial lixiviant in situ.

3. The process of claim 1, wherein the parameter which is measured is indicative of leaching effectiveness.

4. The process of claim 3, wherein the parameter is an electrochemical measurement and/or a temperature measurement.

5. The process of claim 1, comprising the step of taking a baseline measurement of the parameter which is measured and a subsequent measurement following step ii) and comparing the measurements.

6. The process of claim 5, further comprising taking multiple periodic subsequent measurements of the parameter and comparing the measurements.

7. The process of claim 1, wherein multiple parameters are measured.

8. The process of claim 1, wherein the process variable which is altered is the microbial composition of the lixiviant supplied to the body of ore and/or the microbial composition of the lixiviant comprised within the body of ore.

9. The process of claim 1, further comprising the step of contacting the body of metal ore with a second microbial lixiviant having a microorganism composition differing from the microbial lixiviant contacted with the metal ore in step ii).

10. The process of claim 1, wherein the microbial lixiviant and/or the second microbial lixiviant is continuously supplied to the body of metal ore.

11. The process of claim 1, wherein the microbial lixiviant and/or the second microbial lixiviant is intermittently supplied to the body of metal ore.

12. The process of claim 1, wherein the metal content of the metal ore prior to being contacted with microbial lixiviant is about 5000 ppm or less, about 2000 ppm or less, about 1000 ppm or less, about 500 ppm or less, about 200 ppm or less, about 100 ppm or less or about 50 ppm or less.

13. The process of claim 1, wherein the microbial lixiviant and/or the second microbial lixiviant comprises two or more populations of microorganisms.

14. The process of claim 1, wherein the microbial lixiviant comprises at least one, at least two, at least three or at least four populations of microorganisms selected from iron oxidizers, sulfur oxidizers, organic carbon degraders and toxin inactivating microorganisms.

15. A microbial lixiviant composition for use in a bioleaching process, comprising a plurality of strains of microorganisms forming one or more populations of microorganisms selected from the following: iron oxidizers, sulfur oxidizers, organic carbon degraders and toxin inactivating microorganisms.

16. The composition of claim 15, wherein the one, some or all of the populations of microorganisms comprised in the microbial lixiviant are thermally tolerant, acid tolerant, organic carbon tolerant and/or toxin tolerant.

17. The composition of claim 15, wherein the composition comprises one or more nutrients.

18. The composition of claim 15, wherein the microbial lixiviant comprise microorganisms not found together in nature.

19. The composition of claim 15, wherein the microbial lixiviant comprises microorganisms that are present at from about 10² cells/mL to about 10⁸ cells/ml, about 10² cells/mL to about 10⁷ cells/mL or about 10³ cells/mL to about 10⁶ cell/mL

20.The composition of claim 15, wherein the microbial lixiviant comprises one or more of the following populations: (1) iron-oxidizers comprising strains belonging to one or more of the genera Acidocella, Acidimicrobium, Acidiphilium, Acidiplasma, Acidithiobacillus, Acidithiomicrobium, Acidithrix, Ferrithrix, Ferroplasma, Ferrovum, Halomonas, Leptospirillum, Metallosphaera, Mycobacterium, Sulfobacillus, Sulfolobus, Sulfuracidifex and Thermogymnomonas; (2) sulfur oxidizers comprising strains belonging to one or more of the genera Acidianus, Acidicaldus, Acidithiobacillus, Ferviacidithiobacillus, Igneacidithiobacillus, Metallosphaera, Sulfobacillus, Sulfolobus and Sulfuracidifex, (3) organic carbon degraders comprising strains belonging to one or more of the genera Acidicaldus, Acidianus, Acidocella, Acidimicrobium, Acidiphilium, Acidiplasma, Acidimicrobium, Acidithrix, Alicyclobacillus, Ferrithrix, Ferroplasma, Ferrovum, Metallosphaera, Picrophilus, Sulfobacillus, Sulfolobus and Thermoplasma, and (4) Toxin-inactivating microorganisms.

21. The composition of claim 15, wherein one, some or all of the populations of microorganisms in the microbial lixiviant consist of essentially one or more, two or more, or three or more strains of microorganisms.

22. The composition of claim 15, wherein one, some or all of the populations of microorganisms consist of essentially 10 or fewer, 8 or fewer, 5 or fewer or 3 or fewer strains of microorganisms.

23. The composition of claim 15, wherein the microorganisms present in the microbial lixiviant are principally or exclusively not genetically modified microorganisms.

24. A kit comprising a bioreactor apparatus and the composition of claim 15.
